(19) United States
(12) Patent Application Publication
(10) Pub. No.: US 2020/0197239 A1
(43) Pub. Date: Jun. 25, 2020

(54) ABSORBENT ARTICLE WITH IMPROVED FLUID HANDLING

(71) Applicant: Kimberly-Clark Worldwide, Inc., Neenah, WI (US)

(72) Inventors: Francis P. Abuto, John's Creekt, GA (US); Sridhar Ranganathan, Suwanee, GA (US); Charles W. Colman, Marietta, GA (US); Jenny L. Day, Woodstock, GA (US); Vikram Kaul, Atlanta, GA (US)

(21) Appl. No.: 16/645,258

(22) PCT Filed: Sep. 29, 2017
(86) PCT No.: PCT/US2017/054388

§ 371 Date: Mar. 6, 2020

(51) Int. Cl. A61F 13/538

(52) U.S. Cl. CPC A61F 13/538; A61F 2013/530007

(57) ABSTRACT

An absorbent article having a fluid-handling system includes a fluid permeable body side liner; a fluid impermeable outer cover; an absorbent core disposed between the liner and the outer cover, wherein the absorbent core includes superabsorbent material and optionally fluff pulp; a synthetic nonwoven surge layer disposed adjacent the liner between the absorbent core and the liner; and a dispersion layer disposed between the surge layer and the absorbent core, wherein the dispersion layer includes a three-dimensionally patterned, wetlaid, cellulosic tissue nonwoven material. The dispersion layer also includes opposing dispersion layer surfaces each having a textured surface, wherein each surface includes an average material plane, a plurality of ridges extending in a z-direction from the average material plane, and a plurality of grooves alternating with the plurality of ridges, wherein the grooves depth extend in the opposite z-direction from the average material plane.

### BACKGROUND

The present disclosure is generally directed to absorbent articles. Absorbent materials (e.g., surge, absorbent core) are indispensable components of absorbent products such as diapers and pants. Absorbent materials are used extensively to complement an absorbent article's gasketing system by serving as reservoirs in a sense to prevent leakage of body fluids from the products. Although the importance of an effective absorbent system is well recognized, improving the construction and structure of an absorbent system is desired.

Current absorbent articles such as diapers, child training pants, and adult incontinent garments are capacity overdesigned to maintain acceptable leakage performance even though only about one-third of the absorbent core is used when the product is discarded after use. The opportunity cost of the underutilized absorbent core is significant.

The present disclosure provides a solution for increasing absorbent core utilization efficiency. A solution to this problem is important because reducing and/or eliminating leakage, especially early leakage, is critical to delivering a consistently positive experience to the user and the caregiver. The present disclosure addresses these issues by providing an intake system that includes a three-dimensional patterned cellulosic layer.

### SUMMARY

The absorbent products described herein include composites that represent a new class of soft, flexible, and cloth-like nonwoven/film structures that can also potentially be used for a variety of applications such as functional elastics, cleaning wipes, medical fabrics, protection garments, filtration, packaging, and others.

In one aspect, an absorbent article having a fluid-handling system includes a fluid permeable bodyside liner; a fluid impermeable outer cover; an absorbent core disposed between the liner and the outer cover, wherein the absorbent core includes superabsorbent material and optionally fluff pulp; a synthetic nonwoven surge layer disposed adjacent the liner between the absorbent core and the liner; and a dispersion layer disposed between the surge layer and the absorbent core, wherein the dispersion layer includes a three-dimensionally patterned, wetlaid, cellulosic tissue nonwoven material.

In an alternate aspect, an absorbent article having a fluid-handling system includes a fluid permeable bodyside liner; a fluid impermeable outer cover; an absorbent core disposed between the bodyside liner and the outer cover wherein the absorbent core includes at least 5% superabsorbent material and at least 5% fluff pulp; a synthetic nonwoven surge layer disposed adjacent the liner between the absorbent core and the liner; and a dispersion layer disposed between the surge layer and the absorbent core, wherein the dispersion layer includes a three-dimensionally patterned, wetlaid, cellulosic tissue nonwoven material, and wherein the dispersion layer includes opposing dispersion layer surfaces each having a textured surface. Each surface includes an average material plane, a plurality of ridges extending in a z-direction from the average material plane, and a plurality of grooves alternating with the plurality of ridges, wherein the grooves depth extend in the opposite z-direction from the average material plane.

In another aspect, an absorbent article having a fluid-handling system including a fluid permeable bodyside liner; a fluid impermeable outer cover; an absorbent core disposed between the bodyside liner and the outer cover wherein the absorbent core includes at least 5% superabsorbent material and at least 5% fluff pulp; a tissue core wrap encircling the absorbent core; a synthetic nonwoven surge layer disposed adjacent the liner between the absorbent core and the liner; and a dispersion layer disposed between the surge layer and the absorbent core, wherein the dispersion layer includes a three-dimensionally patterned, wetlaid, cellulosic tissue nonwoven material. The dispersion layer includes a basis weight range from about 10 gsm to about 120 gsm, a rush transfer value from about 5% to about 70%, and opposing dispersion layer surfaces each having a textured surface, wherein each surface includes an average material plane, a plurality of ridges extending in a z-direction from the average material plane, and a plurality of grooves alternating with the plurality of ridges, wherein the grooves depth extend in the opposite z-direction from the average material plane, and wherein the grooves having an average depth of about 0.5 mm to about 1 mm and an average frequency of about 0.2 grooves/mm to about 0.5 grooves/mm.

Objects and advantages of the disclosure are set forth below in the following description, or can be learned through practice of the disclosure.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is perspective, partially-cutaway view of a feminine hygiene product of the present disclosure;

FIG. 2 is a perspective view of a particular adult incontinence product of the present disclosure;

FIG. 3 is an elevation schematic view of a cross-section of the adult incontinence product of FIG. 2;

FIG. 4 is a perspective view of an adult absorbent underpant of the present disclosure;

FIG. 5 is a perspective view of a training pant of the present disclosure; and

FIG. 6 is an elevation view of a cross-section of a sheet of UCTAD material, exaggerated to show detail.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

As used herein the term “nonwoven fabric or web” refers to a web having a structure of individual polymeric and/or cellulosic fibers or threads that are interlaid, but not in an identifiable manner as in a knitted fabric. Nonwoven fabrics or webs have been formed from many processes such as for example, meltblowing processes, spunbonding processes, bonded carded web processes, those used to make tissue and towels, etc.

As used herein, the term “meltblown web” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which can be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al., which is incorporated herein in its entirety by reference thereto. Generally speaking, meltblown fibers can be microfibers that are substantially continuous or discontinuous, generally smaller than 10 microns in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 3,692,618 to Dorschner, et al.; U.S. Pat. No. 3,802,817 to Matsuki, et al.; U.S. Pat. No. 3,338,992 to Kinney; U.S. Pat. No. 3,341,394 to Kinney; U.S. Pat. No. 3,502,763 to Hartman; U.S. Pat. No. 3,502,538 to Levy; U.S. Pat. No. 3,542,615 to Dobo, et al.; U.S. Pat. No. 4,340,563 to Appel, et al.; and U.S. Pat. No. 5,382,400 to Pike, et al.; which are incorporated herein in their entirety by reference thereto. Spunbond fibers are generally not tacky when they are deposited onto a collecting surface. Spunbond fibers can sometimes have diameters less than about 40 microns, and are often between about 5 to about 20 microns.

As used herein the term “staple fiber” means fibers that have a fiber length generally in the range of about 0.5 to about 150 millimeters. Staple fibers can be cellulosic fibers or non-cellulosic fibers. Some examples of suitable non-cellulosic fibers that can be used include, but are not limited to, hydrophilically-treated polyolefin fibers, polyester fibers, nylon fibers, polyvinyl acetate fibers, and mixtures thereof. Hydrophilic treatments can include durable surface treatments and treatments in polymer resins/blends. Cellulosic staple fibers include for example, pulp, thermomechanical pulp, synthetic cellulosic fibers, modified cellulosic fibers, and the like. Cellulosic fibers can be obtained from secondary or recycled sources. Some examples of suitable cellulosic fiber sources include virgin wood fibers, such as thermomechanical, bleached and unbleached softwood and hardwood pulps. Secondary or recycled cellulosic fibers can be obtained from office waste, newsprint, brown paper stock, and paperboard scrap. Further, vegetable fibers, such as abaca, flax, milkweed, cotton, modified cotton, cotton linters, can also be used as the cellulosic fibers. In addition, synthetic cellulosic fibers such as, for example, rayon, viscose rayon, and lyocell can be used. Modified cellulosic fibers are generally composed of derivatives of cellulose formed by substitution of appropriate radicals (e.g., carboxyl, alkyl, acetate, nitrate, etc.) for hydroxyl groups along the carbon chain. Desirable staple fibers for the purposes of this application are hydrophilic, such as traditional cellulosic fibers (a desirable example of which is pulp fibers, as can be found in rolled tissues and paper-based towels).

As used herein, the term “substantially continuous fibers” is intended to mean fibers that have a length that is greater than the length of staple fibers. The term is intended to include fibers that are continuous, such as spunbond fibers, and fibers that are not continuous, but have a defined length greater than about 150 millimeters.

As used herein “bonded carded webs” or “BOW” refers to nonwoven webs formed by carding processes as are known to those skilled in the art and further described, for example, in U.S. Pat. No. 4,488,928 to Ali Khan et al., which is incorporated herein by reference thereto. Briefly, carding processes involve starting with a blend of, for example, staple fibers with bonding fibers or other bonding components in a bulky ball that is combed or otherwise treated to provide a generally uniform basis weight. This web is heated or otherwise treated to activate the adhesive component resulting in an integrated, usually lofty nonwoven material.

The basis weight of nonwoven webs is usually expressed in ounces of material per square yard (osy) or grams per square meter (gsm) and fiber diameters are usually expressed in microns, or in the case of staple fibers, denier. It is noted that to convert from osy to gsm, multiply osy by 33.91.

As used herein, the terms “machine direction” or “MD” generally refers to the direction in which a material is produced. It is also often the direction of travel of the forming surface onto which fibers are deposited during formation of a non-woven web. The term “cross-machine direction” or “CD” refers to the direction perpendicular to the machine direction. Dimensions measured in the cross-machine direction (CD) are referred to as “width” dimensions, while dimensions measured in the machine direction (MD) are referred to as “length” dimensions. The width and length dimensions of a planar sheet make up the X and Y directions of the sheet. The dimension in the depth direction of a planar sheet is also referred to as the Z-direction.

As used herein, the term “g/cc” generally refers to grams per cubic centimeter as a measure of density and “cc/g” generally refers to cubic centimeters per gram as a measure of Specific Volume, an inverse of density.

As used herein, the term “hydrophilic” generally refers to fibers or films, or the surfaces of fibers or films that are wettable by aqueous liquids in contact with the fibers. The term “hydrophobic” includes those materials that are not hydrophilic as defined. The phrase “naturally hydrophobic” refers to those materials that are hydrophobic in their chemical composition state without additives or treatments affecting the hydrophobicity.

The degree of wetting of the materials can, in turn, be described in terms of the contact angles and the surface tensions of the liquids and materials involved. Equipment and techniques suitable for measuring the wettability of particular fiber materials or blends of fiber materials can be provided by the Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “composite” as used herein, refers to a film material that has been bonded to or otherwise exists with a nonwoven web including fibers. The film material itself can be mono-layer, multi-component, or multilayer. The composite can be apertured and breathable, or the film material of the composite can be essentially intact.

As used herein, the terms “personal care product” an “absorbent article” refer to any article capable of absorbing water or other fluids. Examples of some absorbent articles include, but are not limited to, personal care absorbent article such as diapers, training pants, absorbent underpants, adult incontinence products including fitted briefs, belted shields, guards for men, protective underwear, adjustable underwear, feminine hygiene products (e.g., sanitary napkins, pad, liners, and the like), swim wear, and so forth. Materials and processes suitable for forming such absorbent articles are well known to those skilled in the art.

Disposable absorbent products are designed to be removed and discarded after a single use. By single use it is meant that the disposable absorbent incontinence product will be disposed of after being used once instead of being laundered or cleaned for reuse, as is typical of regular cloth underwear.

The present disclosure describes personal care products and absorbent products that incorporate an improved fluid handling system. The control of fluid in personal care products is of particular interest to those who use them. The desire to avoid leakage is important to consumers of these products. One aspect of controlling fluid handling addresses the tendency of an absorbent article to become saturated in a target insult area, particularly with multiple insults. Increasing the capability of an absorbent article to move liquid away from the target insult area can help to limit saturation and improve the overall fluid-handling performance of the absorbent article. More specifically, an absorbent article capable of moving fluid from the target insult area, thereby reducing saturation in the target insult area, can improve insult intake, particularly in situations where more than one insult is voided such as third insult intake.

The present disclosure improves absorbent core utilization efficiency, particularly in multiple insult situations, such that less absorbent material is needed, resulting in cost savings. The fluid transport is enabled by using uncreped through-air dried (UCTAD) nonwoven material as a dispersion layer in absorbent articles to distribute fluid from the target insult area. Such a dispersion layer is disposed between the surge layer and the absorbent core. Described herein are ranges of UCTAD properties such as basis weight, textured surface, density, and fiber composition that improve third insult intake time by moving liquid from target insult area.

In various aspects of the disclosure, an absorbent article can include components such as: a liquid-permeable layer (e.g., bodyside liner, surge layer, etc.), a liquid-impermeable layer that can have moisture vapor permeability or breathability (e.g., outer cover, ventilation layer, baffle, etc.), an absorbent core, an elastic member, and so forth. Several examples of such absorbent articles are described in U.S. Pat. No. 5,197,959 to Buell; U.S. Pat. No. 5,085,654 to Buell; U.S. Pat. No. 5,634,916 to Lavon, et al.; U.S. Pat. No. 5,569,234 to Buell, et al.; U.S. Pat. No. 5,716,349 to Taylor, et al.; U.S. Pat. No. 4,950,264 to Osborn, Ill; U.S. Pat. No. 5,009,653 to Osborn, Ill; U.S. Pat. No. 5,509,914 to Osborn, Ill; U.S. Pat. No. 5,649,916 to DiPalma, et al.; U.S. Pat. No. 5,267,992 to Van Tillburg; U.S. Pat. No. 4,687,478 to Van Tillburg; U.S. Pat. No. 4,285,343 to McNair; U.S. Pat. No. 4,608,047 to Mattingly; U.S. Pat. No. 5,342,342 to Kitaoka; U.S. Pat. No. 5,190,563 to Herron, et al.; U.S. Pat. No. 5,702,378 to Widlund, et al.; U.S. Pat. No. 5,308,346 to Sneller, et al.; U.S. Pat. No. 6,110,158 to Kielpikowski; U.S. Pat. No. 6,663,611 to Blaney, et al.; and WO 99/00093 to Patterson, et al.; each of which is incorporated herein in their entirety to the extent they do not conflict herewith.

For purposes of illustration only, certain personal care absorbent products are described herein. This should be considered illustrative only as the absorbent core of the present disclosure can be used in all types of personal care absorbent products including, but not limited to, diapers, training pants, incontinence garments, sanitary napkins, bandages, and the like.

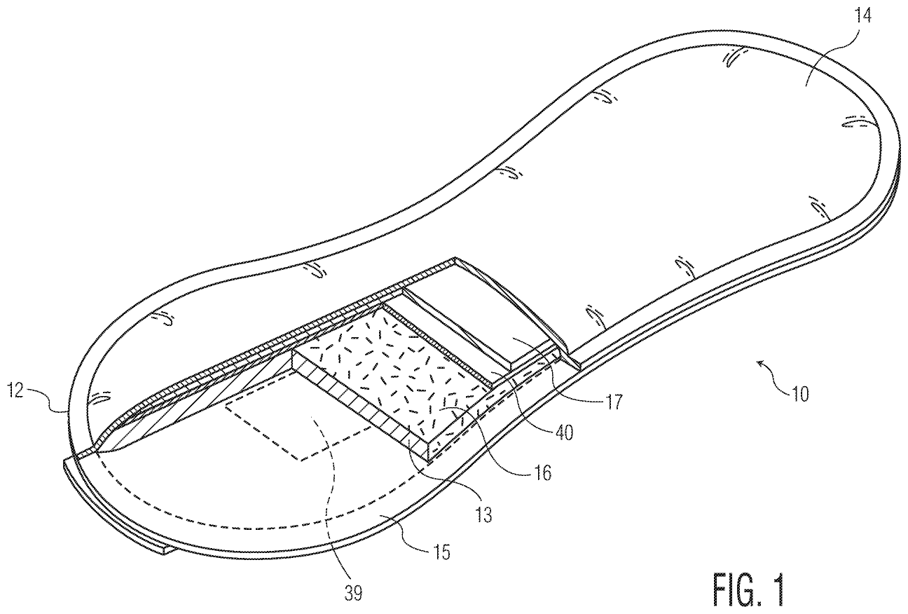

For example, disposable absorbent articles include feminine hygiene pads such as the pad 10 shown in FIG. 1. Pad 10 includes a bodyside liner 14 and a baffle or outer cover 15 that extend to a pad perimeter 12. The pad 10 can include an absorbent core 13 and a transfer or surge layer 17 disposed between the bodyside liner 14 and the baffle or outer cover 15. The absorbent core 13 can include an optional core wrap 16, and is described in more detail below. In an aspect of the present disclosure, the pad 10 can include a dispersion layer 40 positioned between the transfer or surge layer 17 and the absorbent core 13. Many products also have an adhesive strip 39 to help hold the product in place during use by adhering it to the user's underclothes.

Pads typically have a thickness of about 2.5 centimeters (cm) or less. Desirably, the thickness of a pad is less than about 1 cm. More desirably, the thickness of a pad is less than about 0.7 cm. A pad can have a length of from between about 15 cm to about 50 cm, and a width of from between about 2 cm to about 15 cm. Pads can have a rectangular, hourglass, or asymmetrical configuration.

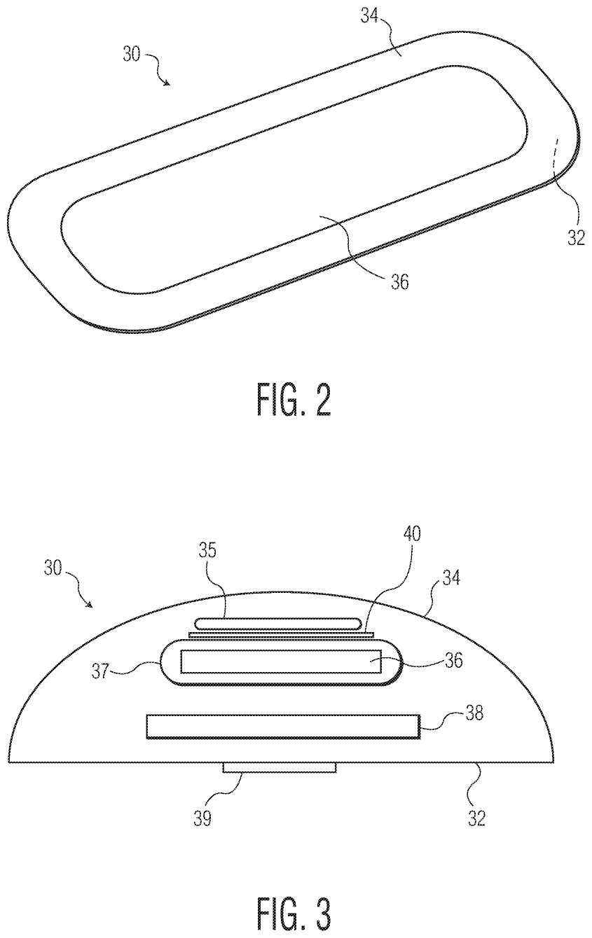

Like feminine hygiene pads, feminine incontinence pads 30 as shown in FIGS. 2 and 3 have a baffle or outer cover 32, a bodyside liner 34, and various layers in between including an absorbent core 36. The absorbent core 36 has a body-facing surface adjacent the bodyside liner 34, a garment-facing surface adjacent the outer-cover 32, and a pair of longitudinal sides. FIG. 3 is a vertical cross-section of one non-limiting example of an incontinence product. The bodyside liner 34 is at the top of FIG. 3. The bodyside liner 34 is designed to allow body fluid, particularly urine, to quickly pass through and be received by an absorbent core 36. The bodyside liner 34 is placed in contact with the genital area of a human body. A surge layer 35 is positioned below the liner 34 and above the absorbent core 36. The surge layer 35 acts as a reservoir to accept large surges of liquid and slowly release them to the subsequent layers. Below the surge layer 35 is the absorbent core 36 surrounded by a substrate in the form of a core wrap 37. Under the substrate-wrapped absorbent core 36 is a baffle or outer cover 32. The absorbent core 36 can include an optional core wrap 37, and is described in more detail below. Further, in one aspect, there is an optional second absorbent layer, such as the airlaid layer 38 seen in FIG. 3. The airlaid layer 38 can be placed either below the core-wrapped absorbent core 36 as shown, or above the core-wrapped absorbent core 36. In an aspect of the present disclosure, the incontinence pad 30 can include a dispersion layer 40 positioned between the surge layer 35 and the absorbent core 36.

A surge layer helps to absorb, decelerate, and diffuse surges or gushes of liquid that may be rapidly introduced into the absorbent article. The surge layer can rapidly accept and temporarily hold the liquid prior to releasing the liquid into, for instance, the absorbent core or any other layer of the absorbent article. The surge layer can be located between the bodyside liner and the absorbent core. Generally, the surge layer can be constructed of any woven or nonwoven material that is easily penetrated by bodily exudates. For example, the surge layer can include a nonwoven fabric layer composed of a meltblown or spunbond web of polyolefin or polyester filaments. Such a nonwoven fabric layer can include conjugate, biconstituent, and homopolymer fibers of staple or other lengths and mixtures of such fibers with other types of fibers. The surge layer can also be a bonded carded web or an airlaid web composed of natural and/or synthetic fibers. The bonded carded web can, for example, be a powder bonded carded web, an infrared bonded carded web, or a through-air bonded carded web. A bonded carded web can optionally include a mixture or blend of different fibers. The surge layer typically has a basis weight of less than about 150 gsm, and in various aspects, from about 10 gsm to about 150 gsm or about 30 gsm to about 150 gsm.

The surge layer can be attached to one or more of various components in the absorbent article such as the absorbent core, the bodyside liner, or the core wrap by methods known in the art, such as by using an adhesive. Examples of suitable surge layers are described in U.S. Pat. Nos. 5,486,166 and 5,490,846. Other suitable surge management materials are described in U.S. Pat. No. 5,820,973. The entire disclosures of these patents are hereby incorporated by reference herein to the extent they are not in conflict herewith.

A pantyliner, not shown, is a relatively thin absorbent pad having a thickness of about 1 cm or less. Desirably, the thickness of a pantyliner is less than about 0.5 cm. A pantyliner can have a length of from between about 15 cm to about 50 cm and a width of from between about 2 cm to about 15 cm. The pantyliner can have a rectangular, hourglass, or asymmetrical configuration and can contain the same components as the pad shown in FIG. 3, or at least the bodyside liner 34, the surge layer 35, the substrate such as core wrap 37, an absorbent core 36, and an outer cover 32.

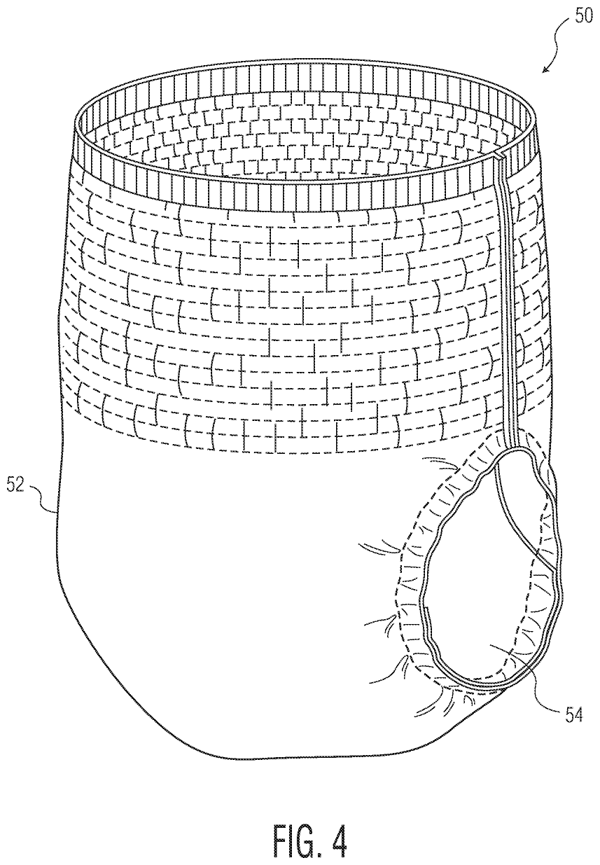

Much of the disposable absorbent incontinence underwear sold today has a unitary configuration that is similar to regular cloth underwear in that the disposable absorbent incontinence underwear is constructed with a waist opening and a pair of leg openings and needs to be pulled onto the body like normal underwear. For example, absorbent underpant 50 as shown in FIG. 4 has an outer cover or baffle 52, a bodyside liner 54, a surge layer (not shown), and an absorbent core (not shown). Further discussion regarding absorbent underpants can be found, for example, in U.S. Pat. No. 6,240,569 to Van Gompel; U.S. Pat. No. 6,367,089 to Van Gompel; and U.S. Patent Publication No. 2004/0210205 A1 to Van Gompel et al.; which are incorporated herein in their entirety by reference thereto to the extent they do not conflict herewith.

Other disposable absorbent incontinence underwear has an open configuration. By an open configuration it is meant that the disposable absorbent incontinence underwear does not have a waist opening and a pair of leg openings before it is positioned about the wearer's torso. Typically, disposable absorbent incontinence underwear having an open configuration has a relatively flat or convex shape before it is secured around the torso of the wearer. Commonly, disposable absorbent incontinence underwear having an open configuration has an approximately rectangular or hourglass shape. Such products are described in U.S. Pat. No. 4,500,316 to Damico, which is incorporated herein in its entirety by reference thereto to the extent it does not conflict herewith.

A belted shield is still another type of a disposable absorbent incontinence product that has an open configuration and is held about the wearer's torso by a belt or a pair of straps, as described in U.S. Pat. No. 5,386,595 to Kuen et al. and U.S. Pat. No. 4,886,512 to Damico et al., which are incorporated herein in their entirety by reference thereto to the extent they do not conflict herewith.

Another type of incontinence product is a guard for men that resembles an absorbent pad that can conform to the male genitalia, as described in U.S. Pat. No. 5,558,659 to Sherrod et al., which is incorporated herein in its entirety by reference thereto to the extent it does not conflict herewith.

More information concerning incontinence products can be found, for example, in U.S. Pat. No. 6,921,393 to Tears et al., which is incorporated herein in its entirety by reference thereto to the extent it does not conflict herewith.

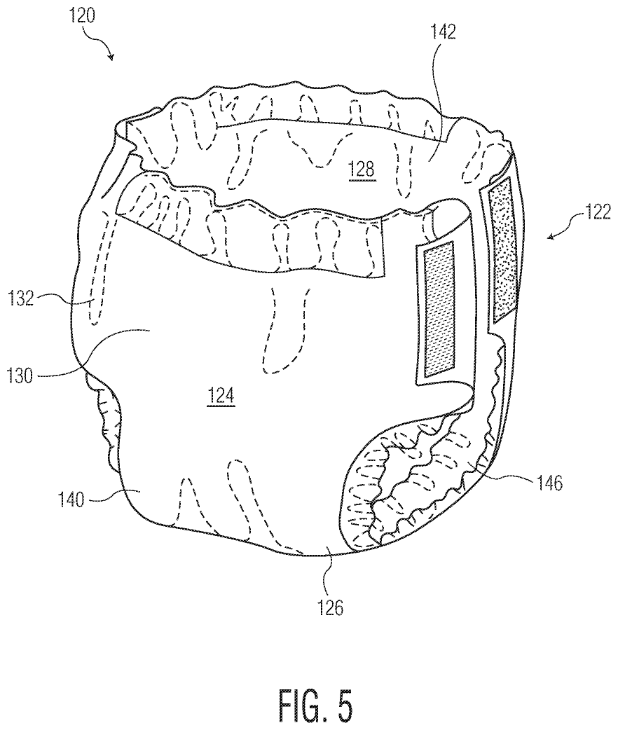

The disposable absorbent article can also be a diaper or training pant, such as the training pant shown in FIG. 5 in a partially fastened condition. The pants 120 define a pair of longitudinal end regions, otherwise referred to herein as a front region 122 and a back region 124, and a center region, otherwise referred to herein as a crotch region 126, extending longitudinally between and interconnecting the front and back regions 122, 124. The pant 120 also defines an inner surface 128 adapted in use (e.g., positioned relative to the other components of the pants 120) to be disposed toward the wearer, and an outer surface 130 opposite the inner surface. The illustrated pants 120 include a chassis 132 that includes an outer cover 140 and a bodyside liner 142 that can be joined to the outer cover 140 in a superimposed relation therewith by adhesives, ultrasonic bonds, thermal bonds or other conventional techniques. The chassis 132 can further include a surge layer (not shown) and an absorbent structure (not shown) disposed between the outer cover 140 and the bodyside liner 142 for absorbing liquid body exudates exuded by the wearer, and can further include a pair of containment flaps 146 secured to the bodyside liner 142 for inhibiting the lateral flow of body exudates.

Disposable absorbent articles generally include an absorbent core or structure as described herein. Each absorbent core typically includes fluff and superabsorbent particles. The superabsorbent particles are loose and very small and therefore can escape onto the body or clothing unless contained. A core wrap (such as the core wrap 37 illustrated in FIG. 3) serves to prevent superabsorbent from migrating from the absorbent core to the user's skin. In FIG. 3 the core wrap 37 is disposed onto the absorbent core 36 by wrapping it at least around the body-facing surface and longitudinal sides of the absorbent core 36. A substrate such as core wrap 37 can be fully wrapped about the absorbent core 36 so that the garment-facing surface is covered as well.

A “superabsorbent or superabsorbent material” refers to a water-swellable, water-soluble organic or inorganic material capable, under the most favorable conditions, of absorbing at least about 20 times its weight and, more desirably, at least about 30 times its weight in an aqueous solution containing 0.9 weight percent sodium chloride. Organic materials suitable for use as a superabsorbent material in conjunction with the present disclosure can include natural materials such as agar, pectin, guar gum, and the like; as well as synthetic materials, such as synthetic hydrogel polymers. Such hydrogel polymers include, for example, alkali metal salts of polyacrylic acids, polyacrylamides, polyvinyl alcohol, ethylene maleic anhydride copolymers, polyvinyl ethers, methyl cellulose, carboxymethyl cellulose, hydroxypropylcellulose, polyvinylmorpholinone; and polymers and copolymers of vinyl sulfonic acid, polyacrylates, polyacrylamides, polyvinylpyrridine, and the like. Other suitable polymers include hydrolyzed acrylonitrile grafted starch, acrylic acid grafted starch, and isobutylene maleic anhydride polymers and mixtures thereof. The hydrogel polymers are preferably lightly crosslinked to render the materials substantially water insoluble. Crosslinking can, for example, be accomplished by irradiation or by covalent, ionic, van der Waals, or hydrogen bonding. The superabsorbent materials can be in any form suitable for use in absorbent composites including particles, fibers, flakes, spheres, and the like. Such superabsorbents are usually available in particle sizes ranging from about 20 to about 1000 microns. The absorbent core 12 can contain from 0 to 100 percent superabsorbent by weight based upon the total weight of the absorbent core.

Typically an absorbent core for a personal care absorbent product will include superabsorbent particles and, optionally, additional absorbent material such as absorbent fibers including, but not limited to, wood pulp fluff fibers, synthetic wood pulp fibers, synthetic fibers and combinations of the foregoing. Wood pulp fluff such as CR-54 wood pulp fluff from Kimberly-Clark Corporation of Neenah, Wis. is an effective absorbent supplement. A common problem with wood pulp fluff, however, is its lack of integrity and its tendency to collapse when wet. As a result, it is often advantageous to add a stiffer reinforcing fiber into the absorbent core such as polyolefin meltblown fibers or shorter length staple fibers. Such combinations of fibers are sometimes referred to as “coform.” The manufacture of meltblown fibers and combinations of meltblown fibers with superabsorbents and/or wood pulp fibers are well known. Meltblown webs are made from fibers formed by extruding a molten thermoplastic material through a plurality of fine, usually circular dye capillaries as molten threads or filaments into a high-velocity heated air stream that attenuates the filaments of molten thermoplastic material to reduce their diameters. Thereafter, the meltblown fibers are carried by the high-velocity gas stream and are deposited on a collecting surface to form a web of randomly dispersed meltblown fibers. The meltblown process is well known and is described in various patents and publications, including NRL Report 4364, “Manufacture of Super-Fine Organic Fibers” by V. A. Wendt, E. L. Boone and C. D. Fluharty; NRL Report 5265, “An Improved Device For the Formation of Super-Fine Thermoplastic Fibers” by K. D. Lawrence, R. T. Lukas and J. A. Young; and U.S. Pat. No. 3,849,241, issued Nov. 19, 1974 to Buntin et al. To form “coform” materials, additional components are mixed with the meltblown fibers as the fibers are deposited onto a forming surface. For example, superabsorbent particles and/or staple fibers such as wood pulp fibers can be injected into the meltblown fiber stream so as to be entrapped and/or bonded to the meltblown fibers. See, for example, U.S. Pat. No. 4,100,324 to Anderson et al.; U.S. Pat. No. 4,587,154 to Hotchkiss et al.; U.S. Pat. Nos. 4,604,313, 4,655,757, and 4,724,114 to McFarland et al.; and U.K. Patent GB 2,151,272 to Minto et al.; all of which are incorporated herein by reference in their entirety.

Referring primarily to FIGS. 1 and 3, the present disclosure is directed to a dispersion layer 40 positioned between a surge or transfer layer and an absorbent core. This design promotes inter-layer fluid flow (within micro-pockets or micro-channels between the nonwoven surge layer and the wetlaid cellulosic dispersion layer) as well as intra-layer fluid flow (wicking within the wetlaid cellulosic dispersion layer) to improve, for example, third fluid insult intake times. Without committing to a theory, the present disclosure uses theoretical principle of channel flow through parallel plates showing that inter-layer fluid flux, i.e. flow between layers, can be as high as 15 to 20 times compared to intra-layer fluid movement, i.e. wicking, within a single material to design and develop the inventive intake system.

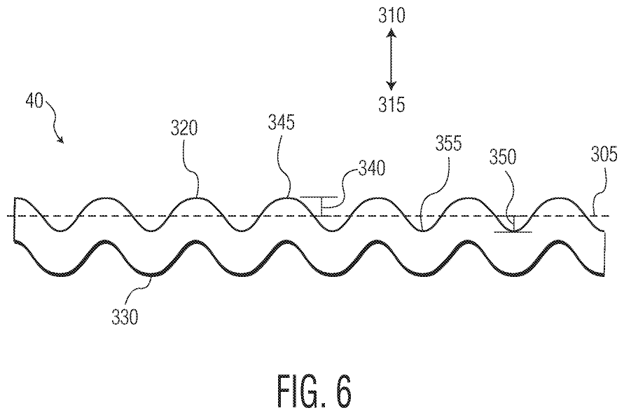

In one particular example, a DEPEND-brand DPU2 female incontinence product has surge material with dimensions of 250 mm×74 mm placed below the bodyside liner approximately 20 mm to 60 mm from the front end of absorbent core. A dispersion layer in the form of a sheet of UCTAD material with the same dimensions was adhesively attached to the surge material such that fluid flows through the bodyside liner, through the surge layer, and then through the dispersion layer before entering the absorbent system (absorbent core and optional core wrap). In other aspects, the dispersion layer 40 can be attached to the surge layer 35 by any suitable means, including ultrasonic attachment, hydroentangling, and the like. This product configuration provides channel spacing between the surge material and the UCTAD dispersion layer that improves, for example, third insult fluid intake time through a combination of inter-layer flow within the channels, and intra-layer wicking within the UCTAD dispersion layer material. This solution creates a channel or tubing using synthetic fibrous web (surge) with relatively open pore structure attached to a wetlaid cellulosic web (UCTAD) wherein the cellulosic web has textured surfaces of micro-channels on both surfaces as shown in FIG. 6. The channels run in a longitudinal direction for fluid transport. The surge provides a temporary fluid source and storage for channel flow between the layers and for the intra-layer wicking within the UCTAD dispersion layer material.

An example of a suitable cellulosic material that can be used as a core wrap is an uncreped through-air dried (UCTAD) sheet having a basis weight of about 15 gsm to about 120 gsm. The UCTAD sheet can be prepared by the process disclosed in U.S. Pat. No. 5,048,589 to Crook et al. and U.S. Pat. No. 5,399,412 to Sudall et al., which are incorporated herein in their entireties to the extent they do not conflict herewith. Broadly, the process includes the steps of forming a furnish of cellulosic fibers, water, and a chemical wet strength resin; depositing the furnish on a traveling foraminous belt thereby forming a fibrous web on top of the traveling belt; subjecting the fibrous web to non-compressive drying to remove water from the fibrous web, and removing the dried fibrous web from the traveling foraminous belt.

FIG. 6 illustrates a cross-section of a portion of a dispersion layer 40 using a sheet of UCTAD material, exaggerated to show detail and therefore not to scale. In a particular aspect, the dispersion layer 40 includes fibers that are entirely natural fibers and preferably entirely cellulose fibers. The dispersion layer 40 preferably has a basis weight range from about 10 gsm to about 250 gsm, and a rush transfer value from about 5% to about 70%. The dispersion layer 40 includes opposing dispersion layer surfaces 320, 330, each having a textured surface. Each surface 320, 330 includes an average material plane 305, a plurality of ridges 345 extending in a z-direction 310 from the average material plane 305, and a plurality of grooves 355 alternating with the plurality of ridges 345, wherein the grooves 355 have a depth extending in the opposite z-direction 315 from the average material plane 305. The grooves 355 have an average depth of about 0.5 mm to about 1 mm and an average frequency of about 0.2 grooves/mm to about 0.5 grooves/mm. The dispersion layer 40 has a longitudinal direction (not shown, into the page), where the grooves 355 extend the full length of the dispersion layer 40 in the longitudinal direction.

The composition of the dispersion layer 40 can be a single material layer or more than one layer stacked on top of each other such that the grooves in the different layers are aligned or misaligned though extending the full length of in the longitudinal direction. Where the dispersion layer 40 includes multiple distinct material layers, the different layers can be affixed to each other such as by adhesive, hydrogen bonding, pressure bonding, or by any suitable means, or the different layers can be unattached to each other. The basis weight of the dispersion layer 40 is greater than about 15 gsm and can be as high as about 250 gsm as a single unitary layer or combined total of multiple layers.

Conventional tissue products are made according to widely known papermaking-type processes. For example, U.S. Pat. No. 5,129,988 to Farrington, Jr.; U.S. Pat. No. 5,772,845 to Farrington, Jr. et al.; and U.S. Pat. No. 5,494,554 to Edwards et al. each disclose various tissue-making methods and are incorporated herein in their entireties by reference thereto to the extent they do not conflict herewith. Current core wraps typically include tissue or a spunbond-meltblown-spunbond (SMS) material.

Reference now will be made in detail to various aspects of the disclosure, one or more examples of which are set forth below. Each example is provided by way of explanation, not of limitation of the disclosure. In fact, it will be apparent to those skilled in the art that various modifications and variations can be made in the present disclosure without departing from the scope or spirit of the disclosure. For instance, features illustrated or described as part of one aspect, can be used on another aspect to yield a still further aspect. Thus it is intended that the present disclosure cover such modifications and variations.

### Cradle Intake Test

First, second, and third intake rates can be determined by the Cradle Intake Test method. This test measures the time required for an absorbent structure to absorb a specific volume of 0.9% saline solution (insult). The absorbent structure is positioned within a test cradle with the intake surface facing up to determine the first intake rate. The insult location is located relative to the transverse center line of the absorbent structure. For products designed for wearers having a weight of 38 to 65 pounds, for example, the insult location is 150 mm forward of the transverse centerline for boys and is 90 mm forward of the transverse centerline for girls and the insult volume is 120 ml for both. For products designed for wearers having a weight of 60 to 120 pounds, in another example, the insult location is 170 mm forward of the transverse centerline for boys and 80 mm forward of the transverse centerline for girls and the insult volume is 220 ml for both. The flow rate of the saline solution is 15 ml/sec and the saline has a temperature of 98.6 degrees F. The absorbent structure is insulted a first time at the aforementioned locations, volumes, and flow rates. The time it takes for the absorbent insert to completely absorb the first insult is recorded. After 15 minutes, the absorbent structure is insulted a second time at the aforementioned locations, volumes, and flow rates. The time it takes for the absorbent structure to completely absorb the second insult is recorded. After 15 minutes, the absorbent structure is insulted a third time at the aforementioned locations, volumes, and flow rates. The time it takes for the absorbent structure to completely absorb the third insult is recorded. The Cradle Intake Test is described in more detail in U.S. Pat. No. 7,977,531 to Dodge, et al., which is incorporated herein to the extent it is relevant and does not conflict herewith.

### Example 1

Example 1 is adult incontinent product, DEPEND-brand DPU 2 female incontinence small/medium absorbent article including an absorbent core disposed between a fluid permeable bodyside liner and a fluid impermeable outer cover. The absorbent core had approximately 19.5 g of superabsorbent material (SAM) and approximately 6.8 g of fluff wrapped in a 16.6 gsm tissue core wrap. The article also included a 100 gsm Bonded Carded Web (BCW) surge material 250 mm long and 74 mm wide disposed between the bodyside liner and the tissue core wrap, approximately 60 mm from the front end of the absorbent core. Attachment adhesive was used between the surge material and the bodyside liner and between the surge material and the tissue core wrap.

Ten products (N=10) were tested in a Cradle Intake Test protocol in which the product was subjected to three (3) insults of 105 mL each at the rate of 8 mL/sec using saline with 0.9% salt concentration, waiting 15 minutes between insults. The average third insult time was 91.04 sec.

### Example 2

Example 2 was the same as Example 1 except that it incorporates the intake system of the present disclosure where the intake system includes an UCTAD dispersion layer of the same dimensions as the surge material, 250 mm long and 74 mm wide, attached to the surge material along its longitudinal edges using approximately 0.06 g of adhesive. This arrangement creates fluid flow channels or tubing between the surge material and the UCTAD dispersion layer. Each channel is capable of expanding from about 2 mm up to about 35 mm in the Z-direction when a squeezing force is applied to the intake system between the fingers in the cross-direction width. The UCTAD sheet was produced with 62% Rush Transfer and approximately 0.3% to 0.9% KYMENE wet strength additive. The UCTAD sheet was a 100% Northern Softwood Kraft (NSWK) pulp fiber composition with a 36 gsm basis weight. The UCTAD sheet included textured surfaces with an average material plane. Each surface included a plurality of ridges extending in the z-direction from the average material plane, and a plurality of grooves alternating with the plurality of ridges, where the depth of the grooves extended in the opposite z-direction from the average material plane. The grooves also extended the full 250 mm length of the UCTAD piece in the longitudinal direction. The grooves had an average depth of about 0.5 mm to about 1 mm and an average frequency of about 0.2 grooves/mm to about 0.5 grooves/mm. The UCTAD sheet also included random pinholes for Z-direction fluid passage to the tissue-wrapped absorbent core.

Ten products (N=10) were tested in a Cradle Test using the same Cradle Intake Test Method as in Example 1. The average third insult intake time was 46.23 sec, providing an improvement of approximately 49% compared to Example 1.

### Example 3

Example 3 was the same as Example 2 except the UCTAD sheet had a blended composition of 60% Eucalyptus and 40% Northern Softwood Kraft (NWSK). The average third insult Cradle Intake time was 52.25 sec, providing an improvement of approximately 43% compared to Example 1.

### Example 4

Example 4 was the same as Example 3 except the UCTAD basis weight was 54 gsm, the fiber composition 100% bleached chemi-thermomechanical pulp (BCTMP), and the Rush Transfer value was 70%. The average third insult Cradle Intake time was 51.56 sec, providing an improvement of approximately 43% compared to Example 1.

### Example 5

Example 5 is the same as Example 4 except the surge basis weight was 50 gsm. The average third insult Cradle Intake time was 65.81 sec, providing an improvement of approximately 28% compared to Example 1.

TABLE 1
Comparing Third Insult Intake Times for control versus UCTAD Dispersion Layer

| | | | | Avg. | Avg. 1st | Avg. 2nd | Avg. 3rd | |
|---|---|---|---|---|---|---|---|---|
| | Basis | | | product | Insult | Insult | Insult | Avg. wet |
| | Wt | Rush | | weight | Intake | Intake | Intake | thickness |
| Example | (gsm) | Transfer | Fiber Blend | (g) | (sec) | (sec) | (sec) | (mm) |
| 1 | n/a | n/a | n/a | 37.17 | 15.75 | 20.79 | 91.04 | 24.66 |
| 2 | 36 | 62 | 100% NSWK | 38.79 | 15.10 | 18.61 | 46.23 | 23.73 |
| 3 | 36 | 62 | Blended60% | 38.37 | 15.75 | 20.01 | 52.25 | 22.77 |
| | | | EUC/40% | | | | | |
| | | | NSWK | | | | | |
| 4 | 54 | 70 | 100% BCTMP | 38.60 | 15.06 | 18.07 | 51.56 | 22.33 |
| 5 | 54 | 70 | 100% BCTMP | 38.70 | 15.33 | 19.98 | 65.81 | 21.97 |

By moving large amounts of fluid from the target area, the UCTAD dispersion layer material enables the absorbent article to intake multiple fluid insults at a faster rate because the target area is less saturated for the incoming third insult, for example. The structure of UCTAD has more channels and space for fluid to move through. UCTAD materials when compared to conventional tissue materials will have a higher air permeability, or z-plane direction permeability. Another benefit of UCTAD is the potential for fluid to wick in the x-y plane to increase core utilization and decrease weight in the product's target zone.

Certain properties of the wetlaid UCTAD cellulosic webs can be adjusted for use as a dispersion layer in absorbent articles to improve third insult intake time. The intake time is improved through a careful selection and control of UCTAD material fiber composition, basis weight, specific volume, surface texture, and process parameters such as Rush Transfer to enable transport and movement of fluid from the absorbent article insult target area.

Textured surfaces that help with fluid channeling and distribution are particularly beneficial in this disclosure. As illustrated in Table 2, the textured surface grooves typically have an average depth of about 0.5 mm to about 1 mm and a frequency of about 0.2 grooves/mm to about 0.5 grooves/mm, though smaller or larger dimensions and or frequencies are contemplated.

All else being equal, for example by keeping basis weight and rush transfer of the core wrap constant, and without being held to any theory, deeper grooves corresponding to higher ridges (see FIG. 6) appear to provide channels with greater capacity for transporting greater amounts of liquid volumes inter-layer. Similarly, higher specific volume (inverse of density) can provide inter-fiber void space capacity for greater amounts of fluid transport intra-layer.

TABLE 2
Groove Height and Spacing Measurements for UCTAD Codes

| | |
|---|---|
| Outside | Inside |

| Measurement | avg. | std. dev. | avg. | std. dev. |
|---|---|---|---|---|
| Frequency (mm⁻¹) | 0.32 | 0.02 | 0.33 | 0 |
| Height (mm) | 0.74 | 0.05 | 0.8 | 0.17 |
| Spacing (mm) | 3.65 | 0.36 | 3.22 | 0.27 |

In a first particular aspect, an absorbent article having a fluid-handling system includes a fluid permeable bodyside liner; a fluid impermeable outer cover; an absorbent core disposed between the liner and the outer cover, wherein the absorbent core includes superabsorbent material and optionally fluff pulp; a synthetic nonwoven surge layer disposed adjacent the liner between the absorbent core and the liner; and a dispersion layer disposed between the surge layer and the absorbent core, wherein the dispersion layer includes a three-dimensionally patterned, wetlaid, cellulosic tissue nonwoven material.

A second particular aspect includes the first particular aspect, further including a tissue core wrap encircling the absorbent core.

A third particular aspect includes the first and/or second aspect, wherein the three-dimensionally patterned, cellulosic tissue nonwoven material is an uncreped, through-air dried (UCTAD) material.

A fourth particular aspect includes one or more of aspects 1-3, wherein the dispersion layer has a basis weight range from about 10 gsm to about 120 gsm.

A fifth particular aspect includes one or more of aspects 1-4, wherein the dispersion layer is produced using a rush transfer value from about 5% to about 70%.

A sixth particular aspect includes one or more of aspects 1-5, wherein the dispersion layer includes opposing dispersion layer surfaces each having a textured surface, wherein each surface includes an average material plane, a plurality of ridges extending in a z-direction from the average material plane, and a plurality of grooves alternating with the plurality of ridges, and wherein the grooves have a depth extending in the opposite z-direction from the average material plane.

A seventh particular aspect includes one or more of aspects 1-6, wherein the grooves have an average depth greater than 0.1 mm.

An eighth particular aspect includes one or more of aspects 1-7, wherein the grooves have an average depth of about 0.5 mm to about 1 mm.

A ninth particular aspect includes one or more of aspects 1-8, wherein the grooves have an average frequency of about 0.2 grooves/mm to about 0.5 grooves/mm.

A tenth particular aspect includes one or more of aspects 1-9, wherein the dispersion layer has a longitudinal direction, and wherein the grooves extend the full length of the dispersion layer in the longitudinal direction.

An eleventh particular aspect includes one or more of aspects 1-10, wherein a third fluid insult intake time is at least 50 percent faster than that of the same article without a dispersion layer.

A twelfth particular aspect includes one or more of aspects 1-11, wherein the article is a diaper, a training pant, an adult incontinence product, or a feminine hygiene product.

A thirteenth particular aspect includes one or more of aspects 1-12, wherein the dispersion layer is affixed to the surge layer.

In a fourteenth particular aspect, an absorbent article having a fluid-handling system includes a fluid permeable bodyside liner; a fluid impermeable outer cover; an absorbent core disposed between the bodyside liner and the outer cover wherein the absorbent core includes at least 5% superabsorbent material and at least 5% fluff pulp; a synthetic nonwoven surge layer disposed adjacent the liner between the absorbent core and the liner; and a dispersion layer disposed between the surge layer and the absorbent core, wherein the dispersion layer includes a three-dimensionally patterned, wetlaid, cellulosic tissue nonwoven material, and wherein the dispersion layer includes opposing dispersion layer surfaces each having a textured surface, wherein each surface includes an average material plane, a plurality of ridges extending in a z-direction from the average material plane, and a plurality of grooves alternating with the plurality of ridges, wherein the grooves depth extend in the opposite z-direction from the average material plane.

A fifteenth particular aspect includes the fourteenth particular aspect, wherein the grooves having an average depth of about 0.5 mm to about 1 mm and an average frequency of about 0.2 grooves/mm to about 0.5 grooves/mm.

A sixteenth particular aspect includes the fourteenth and/or fifteenth aspect, wherein the dispersion layer includes a basis weight range from about 10 gsm to about 120 gsm, a rush transfer value from about 5% to about 70%, and opposing dispersion layer surfaces each having a textured surface.

A seventeenth particular aspect includes one or more of aspects 14-16, wherein the dispersion layer is an uncreped, through-air dried (UCTAD) material.

In an eighteenth particular aspect, an absorbent article having a fluid-handling system includes a fluid permeable bodyside liner; a fluid impermeable outer cover; an absorbent core disposed between the bodyside liner and the outer cover wherein the absorbent core includes at least 5% superabsorbent material and at least 5% fluff pulp; a tissue core wrap encircling the absorbent core; a synthetic nonwoven surge layer disposed adjacent the liner between the absorbent core and the liner; and a dispersion layer disposed between the surge layer and the absorbent core, wherein the dispersion layer includes a three-dimensionally patterned, wetlaid, cellulosic tissue nonwoven material, wherein the dispersion layer includes a basis weight range from about 10 gsm to about 120 gsm, a rush transfer value from about 5% to about 70%, and opposing dispersion layer surfaces each having a textured surface, wherein each surface includes an average material plane, a plurality of ridges extending in a z-direction from the average material plane, and a plurality of grooves alternating with the plurality of ridges, wherein the grooves depth extend in the opposite z-direction from the average material plane, and wherein the grooves having an average depth of about 0.5 mm to about 1 mm and an average frequency of about 0.2 grooves/mm to about 0.5 grooves/mm.

A nineteenth particular aspect includes the eighteenth particular aspect, wherein the dispersion layer has a longitudinal direction, and wherein the grooves extend the full length of the dispersion layer in the longitudinal direction.

A twentieth particular aspect includes the eighteenth and/or nineteenth particular aspects, wherein the dispersion layer is an uncreped, through-air dried (UCTAD) material.

While the disclosure has been described in detail with respect to the specific aspects thereof, it will be appreciated that those skilled in the art, upon attaining an understanding of the foregoing, can readily conceive of alterations to, variations of, and equivalents to these aspects. Accordingly, the scope of the present disclosure should be assessed as that of the appended claims and any equivalents thereto.

What is claimed is:

1. An absorbent article having a fluid-handling system, the article comprising:
a fluid permeable bodyside liner;
a fluid impermeable outer cover;
an absorbent core disposed between the liner and the outer cover, wherein the absorbent core includes superabsorbent material and optionally fluff pulp;
a synthetic nonwoven surge layer disposed adjacent the liner between the absorbent core and the liner; and
a dispersion layer disposed between the surge layer and the absorbent core, wherein the dispersion layer includes a three-dimensionally patterned, wetlaid, cellulosic tissue nonwoven material.

2. The article of claim 1, further comprising a tissue core wrap encircling the absorbent core.

3. The article of claim 1, wherein the three-dimensionally patterned, cellulosic tissue nonwoven material is an uncreped, through-air dried (UCTAD) material.

4. The article of claim 1, wherein the dispersion layer has a basis weight range from about 10 gsm to about 120 gsm.

5. The article of claim 1, wherein the dispersion layer is produced using a rush transfer value from about 5% to about 70%.

6. The article of claim 1, wherein the dispersion layer includes opposing dispersion layer surfaces each having a textured surface, wherein each surface includes an average material plane, a plurality of ridges extending in a z-direction from the average material plane, and a plurality of grooves alternating with the plurality of ridges, and wherein the grooves have a depth extending in the opposite z-direction from the average material plane.

7. The article of claim 6, wherein the grooves have an average depth greater than 0.1 mm.

8. The article of claim 6, wherein the grooves have an average depth of about 0.5 mm to about 1 mm.

9. The article of claim 6, wherein the grooves have an average frequency of about 0.2 grooves/mm to about 0.5 grooves/mm.

10. The article of claim 6, wherein the dispersion layer has a longitudinal direction, and wherein the grooves extend the full length of the dispersion layer in the longitudinal direction.

11. The article of claim 1, wherein a third fluid insult intake time is at least 50 percent faster than that of the same article without a dispersion layer.

12. The article of claim 1, wherein the article is a diaper, a training pant, an adult incontinence product, or a feminine hygiene product.

13. The article of claim 1, wherein the dispersion layer is affixed to the surge layer.

14. An absorbent article having a fluid-handling system, the article comprising:
a fluid permeable bodyside liner;
a fluid impermeable outer cover;
an absorbent core disposed between the bodyside liner and the outer cover wherein the absorbent core comprises at least 5% superabsorbent material and at least 5% fluff pulp;
a synthetic nonwoven surge layer disposed adjacent the liner between the absorbent core and the liner; and
a dispersion layer disposed between the surge layer and the absorbent core, wherein the dispersion layer includes a three-dimensionally patterned, wetlaid, cellulosic tissue nonwoven material, and wherein the dispersion layer includes opposing dispersion layer surfaces each having a textured surface, wherein each surface includes
an average material plane,
a plurality of ridges extending in a z-direction from the average material plane, and
a plurality of grooves alternating with the plurality of ridges, wherein the grooves depth extend in the opposite z-direction from the average material plane.

15. The article of claim 14, wherein the grooves having an average depth of about 0.5 mm to about 1 mm and an average frequency of about 0.2 grooves/mm to about 0.5 grooves/mm.

16. The article of claim 14, wherein the dispersion layer comprises:
a basis weight range from about 10 gsm to about 120 gsm,
a rush transfer value from about 5% to about 70%, and
opposing dispersion layer surfaces each having a textured surface.

17. The article of claim 14, wherein the dispersion layer is an uncreped, through-air dried (UCTAD) material.

18. An absorbent article having a fluid-handling system, the article comprising:
a fluid permeable bodyside liner;
a fluid impermeable outer cover;
an absorbent core disposed between the bodyside liner and the outer cover wherein the absorbent core comprises at least 5% superabsorbent material and at least 5% fluff pulp;
a tissue core wrap encircling the absorbent core;
a synthetic nonwoven surge layer disposed adjacent the liner between the absorbent core and the liner; and
a dispersion layer disposed between the surge layer and the absorbent core, wherein the dispersion layer includes a three-dimensionally patterned, wetlaid, cellulosic tissue nonwoven material, wherein the dispersion layer comprises:
a basis weight range from about 10 gsm to about 120 gsm,
a rush transfer value from about 5% to about 70%, and
opposing dispersion layer surfaces each having a textured surface, wherein each surface includes an average material plane, a plurality of ridges extending in a z-direction from the average material plane, and a plurality of grooves alternating with the plurality of ridges, wherein the grooves depth extend in the opposite z-direction from the average material plane, and wherein the grooves having an average depth of about 0.5 mm to about 1 mm and an average frequency of about 0.2 grooves/mm to about 0.5 grooves/mm.

19. The article of claim 18, wherein the dispersion layer has a longitudinal direction, and wherein the grooves extend the full length of the dispersion layer in the longitudinal direction.

20. The article of claim 18, wherein the dispersion layer is an uncreped, through-air dried (UCTAD) material.
